(19) United States
(12) Patent Application Publication
(10) Pub. No.: US 2026/0069735 A1
(43) Pub. Date: Mar. 12, 2026

(54) PREPARATION METHOD OF CALCIUM-BASED METAL-ORGANIC FRAMEWORK FOR SELECTIVELY ADSORBING FISHY OFF-ODOR COMPOUNDS AND USES THEREOF

(71) Applicants: Huazhong Agricultural University, Wuhan (CN); Dalian Polytechnic University, Dalian (CN)

(72) Inventors: Huang Qilin, Wuhan (CN); Li Haiyan, Wuhan (CN); Luo Hongmei, Wuhan (CN); Wu Dan, Wuhan (CN)

(73) Assignees: Huazhong Agricultural University, Wuhan, HB (CN); Dalian Polytechnic University, Dalian, LN (CN)

(21) Appl. No.: 19/315,895

(22) Filed: Sep. 2, 2025

(30) Foreign Application Priority Data
Sep. 10, 2024 (CN) 202411265111.5

(51) Int. Cl. A61L 9/014; B01J 20/22; B01J 20/30

(52) U.S. Cl. CPC A61L 9/014; B01J 20/226; B01J 20/3071; B01J 2220/4812

(57) ABSTRACT

A method for preparing a calcium-based metal-organic framework (Ca-MOF) for selectively adsorbing fishy off-odor compounds is disclosed. The method involves dissolving an organic acid and a calcium salt in an organic solvent, then transferring the solution to a high-pressure reactor for a high-temperature reaction. The solution is subsequently cooled to room temperature, followed by centrifuging, alcohol washing, and drying to yield Ca-MOF crystals. The resulting Ca-MOF demonstrates a selective and efficient adsorption of sulfur-containing compounds present in fishy off-odors, achieving up to a 97.3% removal efficiency for dimethyl trisulfide. With a fast adsorption rate, this Ca-MOF can be applied directly to food or used in an external sachet, making it suitable for diverse applications in food, medicine, and pesticides.

### TECHNICAL FIELD

The present disclosure belongs to the technical field of metal-organic framework materials, and relates to a preparation method of a calcium-based metal-organic framework (Ca-MOF) for selectively adsorbing fishy off-odor compounds and uses thereof.

### BACKGROUND

As one of the three major categories of animal-derived foods alongside livestock and poultry meat and eggs, aquatic products are renowned for their delicious flavor, provide high-quality protein essential to the human body, and are favored by consumers for their high nutritional value and low calorie content. The aquatic products derived therefrom have high economic value. However, they are susceptible to lipid oxidation, enzymatic reactions, and microbial degradation, which cause fish meat spoilage, protein degradation, and the generation of off-odors during processing and storage, greatly impairing the consumption and processing of aquatic products.

The fishy odor of aquatic products is mainly attributed to substances such as hexanal, nonanal, 4-heptenal, 2,4-heptadienal, and 1-octen-3-ol produced by lipid oxidation; nitrogen-containing and sulfur-containing substances generated from protein degradation; and geosmin and 2-methylisoborneol produced by microbial metabolism in the aquatic environment. Among these, sulfur-containing compounds with garlic-like and rotten fish odors, due to their high content and low threshold, contribute significantly to the fishy odor but are extremely difficult to remove. Thus, the problem of strong and hard-to-eliminate fishy odor in aquatic products has become a bottleneck restricting the quality improvement and industrial development of aquatic products.

### SUMMARY

The present disclosure provides a preparation method of a calcium-based metal-organic framework (Ca-MOF) for selectively adsorbing fishy off-odor compounds, and uses thereof. By utilizing a high specific surface area and unsaturated metal sites, the Ca-MOF adsorbs and removes fishy odors from aquatic products, thereby solving the problems of strong and persistent fishy odors in aquatic products. The preparation process is simple, the adsorption performance is excellent, and the material has great application prospects.

To achieve the above objective, the present disclosure adopts the following technical solutions:
A preparation method of a Ca-MOF for selectively adsorbing fishy off-odor compounds, the method including the following steps:
S1. dissolving an organic acid and a calcium salt in an organic solvent;
S2. transferring a dissolved solution into a reactor for a high-temperature reaction, and then cooling to room temperature; and
S3. performing centrifuging, alcohol washing, and drying on the cooled solution to obtain Ca-MOF crystals.

Preferably, the organic acid in step S1 is one or a combination of fumaric acid, succinic acid, lactic acid, oxalic acid, citric acid, malic acid, benzoic acid, cinnamic acid, caffeic acid, ferulic acid, p-coumaric acid, sinapic acid, and syringic acid.

Preferably, the calcium salt in step S1 is one or a combination of calcium acetate, calcium carbonate, calcium chloride, calcium nitrate, calcium phosphate, and calcium hydrogen phosphate, and a molar ratio of the organic acid to the calcium salt is 1: (1-5).

Preferably, the organic acid in step S1 is subjected to modification with an ammonium salt before use, and the ammonium salt is one or a combination of ammonium bicarbonate, ammonium carbonate, ammonium chloride, ammonium phosphate, and ammonium hydrogen phosphate, where a modification method comprises dissolving the ammonium salt and the organic acid in a saturated NaCl solution, with a molar ratio of the ammonium salt to the organic acid of 1: (1-5); and after forming a monoammonium salt of the organic acid, dissolving the monoammonium salt of the organic acid and the calcium salt together in an organic solvent, where the organic solvent is methanol or ethanol, with a volume ratio of the organic solvent to water is 1: (1-5).

Further preferably, an ammonium salt is further capable of being added to the organic acid and the calcium salt in step S1, where the ammonium salt is one or a combination of ammonium bicarbonate, ammonium carbonate, ammonium chloride, ammonium phosphate, and ammonium hydrogen phosphate, and a molar ratio of the ammonium salt to the organic acid to the calcium salt is 1: (1-5): (1-5).

Preferably, a high-temperature reaction condition in step S2 includes: placing the high-pressure reactor at 60° C. to 300° C. for a reaction time of 6 to 72 h.

Preferably, in step S3, alcohol washing is performed 1 to 5 times using ethanol or methanol, and drying conditions include vacuum drying at 20° C. to 100° C. for 1 to 24 h under a pressure of −0.01 to −0.1 MPa.

The Ca-MOF with selective adsorption obtained by the preparation method can be used for compounding with a γ-cyclodextrin metal-organic framework, and a composite supramolecular MOF is prepared by a nucleation kinetics-directed growth method, and then used for adsorption.

The Ca-MOF with selective adsorption obtained by the preparation method can be used for adsorbing sulfur-containing compounds, aldehydes, alcohols, nitrogen-containing compounds, heterocyclic compounds, or other substances, and can be used in external adsorption sachets or directly added to food.

Preferably, the Ca-MOF with selective adsorption obtained by the preparation method is used for gas separation and purification in the chemical industry, or adsorption and removal of heavy metals and pesticides in three-waste treatment, and detection of toxins, heavy metals, or pesticides as a biological or electrochemical sensor, or used as a drug carrier in the pharmaceutical industry.

Compared with the prior art, the present disclosure has the advantages and beneficial effects as follows:
(1) The Ca-MOF prepared in the present disclosure has the following three advantages: first, it can efficiently adsorb various undesirable fishy off-odor compounds; second, Ca²⁺ can enhance the gel strength of surimi and inhibit the release of fishy odors; third, it introduces Ca, an essential macroelement for the human body.
(2) The Ca-MOF prepared in the present disclosure is applicable in various scenarios. It can not only be directly added to food but also be used as an external adsorption bag to effectively remove various off-odor compounds.
(3) The Ca-MOF prepared in the present disclosure is structurally characterized using a powder X-ray diffractometer, a specific surface area and porosity analyzer, and a Fourier transform infrared spectrometer. The results show that the Ca-MOF is successfully prepared and has a high specific surface area (600-1200 m²/g).
(4) The adsorption experiment results of the present disclosure show that the Ca-MOF exhibited significant selective adsorption capacity for dimethyl trisulfide (a sulfur-containing compound), with an adsorption efficiency of up to 97.3%, and adsorption equilibrium can be achieved within 30 min.
(5) The functionalized and modified Ca-MOF prepared in the present disclosure can significantly improve the adsorption performance of sulfur-containing substances due to the increased polar adsorption sites.
(6) The composite supramolecular MOF material prepared in the present disclosure has microporous, mesoporous, and macroporous structures, which greatly expand the adsorption range of adsorbents and can significantly improve the adsorption capacity for various compounds.
(7) The present invention integrates food science, material synthesis technology, and physical adsorption principles to develop a Ca-MOF with selective adsorption. It not only expands the application of metal-organic frameworks in the food field but also provides a green and safe porous material that can be applied in multiple fields including chemical industry, three-waste treatment, pharmaceuticals, and food safety testing.

### DESCRIPTION OF THE DRAWINGS

FIG. 1 shows a flow chart of a preparation method of a Ca-MOF for selectively adsorbing fishy off-odor compounds according to the present disclosure;

FIG. 2 shows a powder X-ray diffraction pattern of the Ca-MOF according to Example 1 of the present disclosure;

FIG. 3 shows an FTIR spectrum of the Ca-MOF according to Example 1 of the present disclosure;

FIG. 4 shows a N2 adsorption-desorption isotherm curve of the Ca-MOF according to Example 1 of the present disclosure;

FIG. 5 shows a pore diameter distribution diagram of the Ca-MOF according to Example 1 of the present disclosure;

FIG. 6 shows a powder X-ray diffraction pattern of Example 1 and comparative Examples of the present disclosure; and

FIG. 7 shows Fourier transform infrared spectra of Example 1 and comparative Examples of the present disclosure.

### DETAILED DESCRIPTION OF EMBODIMENTS

In order to make objectives, technical solutions, and advantages of the present disclosure clearer, the present disclosure is described in further detail below with reference to drawings and embodiments. It should be understood that the specific embodiments described herein are only intended to explain the present disclosure, but not to limit the present disclosure. In addition, the technical features involved in the various embodiments of the present disclosure described below can be combined with each other as long as they do not conflict with one another. It is clear that the embodiments described are part of the embodiments of the present disclosure, but not all of them. Based on the embodiments of the present disclosure, all other embodiments obtained by those of ordinary skill in the art without creative efforts shall fall within the scope of protection of the present disclosure.

### Example 1

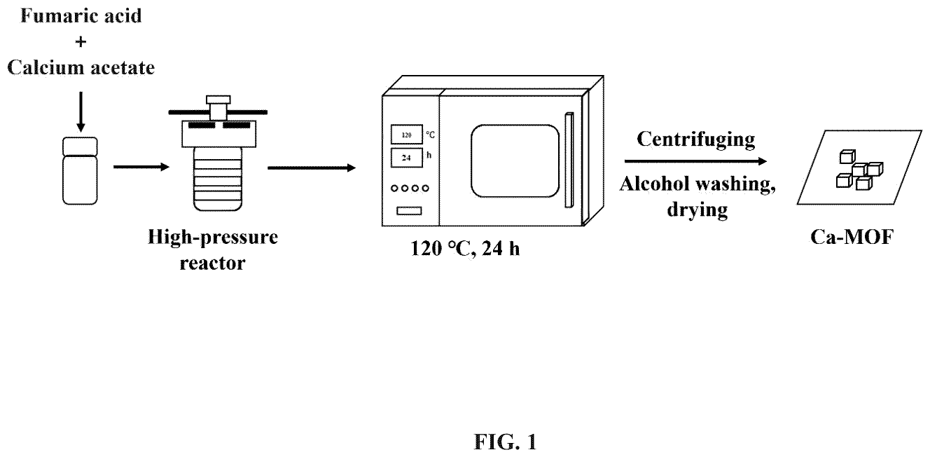

The specific process for preparing the Ca-MOF with selective adsorption is as follows: referring to FIG. 1, accurately weighing 5 g of fumaric acid and 5 g of calcium acetate, dissolving them in a mixed solution of methanol (10 mL) and ultrapure water (20 mL), and fully dissolving under magnetic stirring; and transferring the solution into a polytetrafluoroethylene high-pressure reactor, reacting at 120° C. for 24 h, cooling to room temperature, collecting white crystals by centrifugation, washing 3 times with methanol, and drying under vacuum at 60° C. for 4 h to obtain Ca-MOF1.

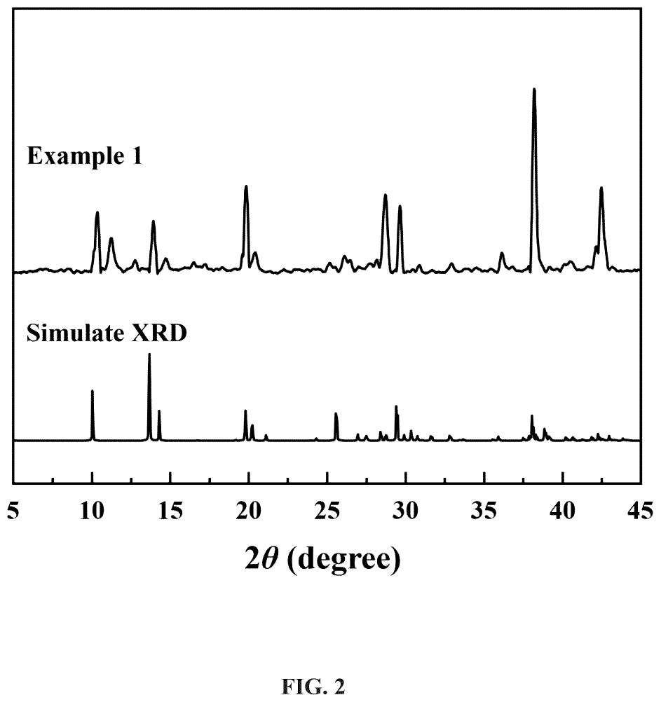

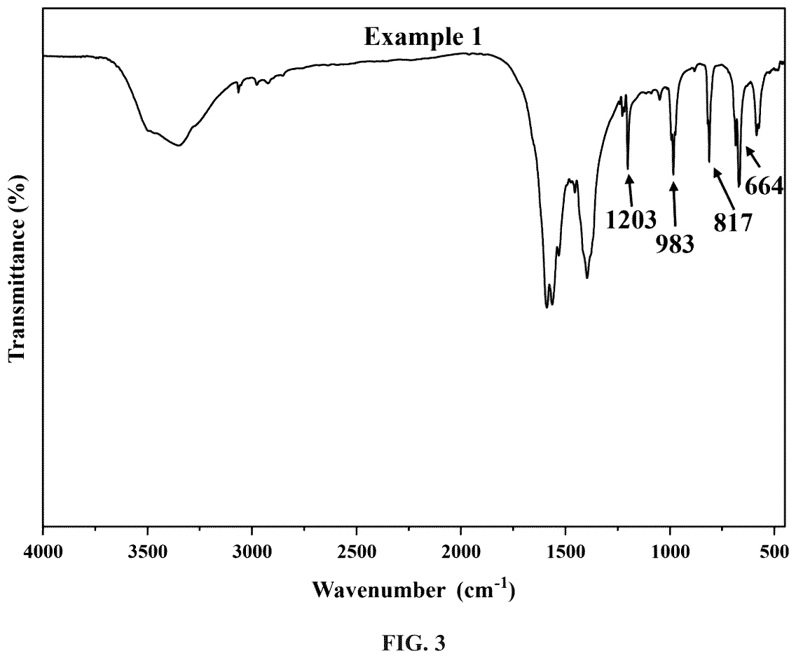

FIG. 2 shows an XRD pattern of the Ca-MOF1, showing that the Ca-MOF1 has a good crystalline structure with sharp diffraction peaks appearing at 10.3°, 13.9°, 14.6°, 19.8°, 28.6°, and 38.2° respectively, which is consistent with an XRD spectrum simulated by Materials Studio software; FIG. 3 is the FTIR spectrum of the Ca-MOF1, showing absorption peaks at 3465, 1530, and 1407 cm-1, which are attributed to O—H stretching vibration and symmetric and asymmetric stretching vibrations of C═O, respectively. The sharp and strong absorption peaks at 1203, 983, and 817 cm-1 indicate the coordination of carboxyl groups in fumaric acid with Ca, and the absorption peak at 664 cm-1 is attributed to the stretching vibration of Ca—O bonds. The XRD and FTIR results confirm the successful synthesis of a Ca-MOF material.

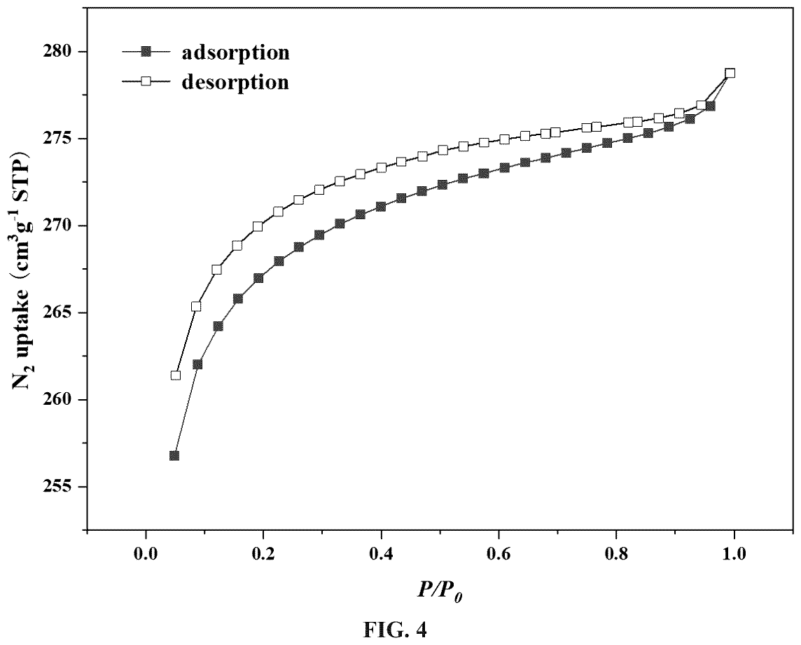

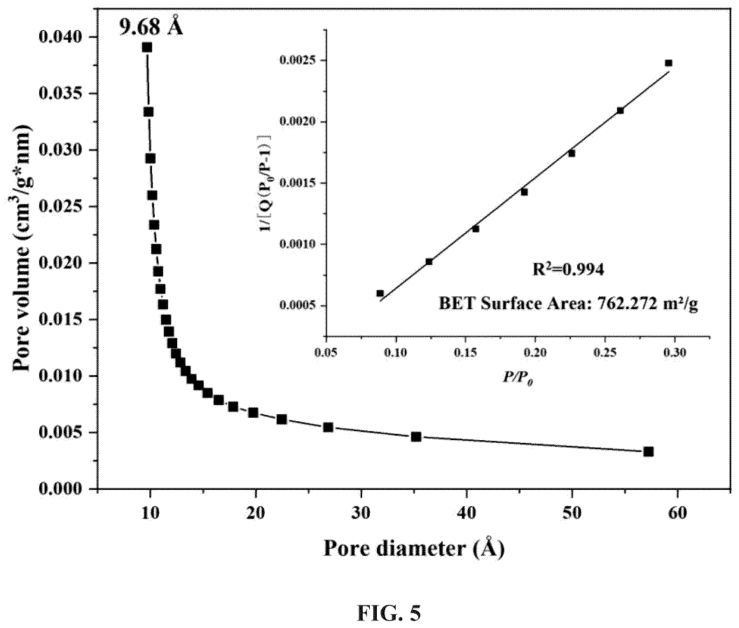

Combined with the N2 adsorption-desorption isotherm curve in FIG. 4 and the pore diameter distribution diagram in FIG. 5, it is shown that the Ca-MOF1 has a specific surface area of 762.272 m2/g and an average pore diameter of 9.68 Å.

Experimental verification of deodorization application in this Example:
(1) Static adsorption experiment:

Standard samples of characteristic fishy off-odor compounds, such as nonanal, 1-octen-3-ol, dimethyl trisulfide, trimethylamine, and 2-methylisoborneol, were first dissolved in methanol. According to the proportion of odor activity values (OVA values) of different substances in surimi, they were diluted with ultrapure water to required concentrations (nonanal 150 μg/mL, 1-octen-3-ol 350 μg/mL, dimethyl trisulfide 200 μg/mL, trimethylamine 500 μg/mL, and 2-methylisoborneol 15 μg/mL). 1 g of Ca-MOF1 was weighed and added to 2 mL of a mixed fishy off-odor compound solution. The mixture was sealed, placed in a magnetic heating stirrer, and then stirred at a low speed of 100 rpm at 40° C. After 60 min, the mixture was centrifuged, and 1 mL of the supernatant was taken. The concentration Cₜ of the fishy off-odor compounds in a filtrate was measured using solid-phase microextraction-gas chromatography (SPME-GC). The adsorption capacity qt (μg/g) and adsorption efficiency Qi of the Ca-MOF1 for the fishy off-odor compounds at time t were calculated according to the following formulas:

$$Adsorption capacity for fishy off - odor compounds at time t :$$

$${q}_{t} = \frac{( {C}_{0} - {C}_{t} ) V}{m}$$

$$Adsorption efficiency : {Q}_{i} = \frac{( {C}_{0} - {C}_{t} )}{{C}_{0}} \times 100 \%$$

where C₀ and Cₜ are the initial concentration of the fishy off-odor compounds solution and the concentration of fishy-smelling substances in the solution at time t, respectively; V is the volume of the solution; m is the mass of the Ca-MOF1. Table 1 shows the equilibrium adsorption capacity and the adsorption efficiency of the Ca-MOF1 for adsorbing five types of fishy off-odor compounds.

TABLE 1

| | Equilibrium adsorption | Adsorption |
|---|---|---|
| | capacity (μg/g) | efficiency (%) |
| Nonanal | 1564.61 | 85.7 |
| 1-Octen-3-ol | 1209.72 | 47.1 |
| Dimethyl trisulfide | 2270.70 | 97.3 |
| Trimethylamine | 8936.26 | 79.2 |
| 2-Methylisoborneol | 220.01 | 42.5 |

As shown in Table 1, the Ca-MOF1 exhibits strong adsorption capacity for aldehydes, dimethyl trisulfide, and trimethylamine, and shows highly selective adsorption capacity (an adsorption efficiency of up to 97.3%) for dimethyl trisulfide (a sulfur-containing substance). This may be attributed to the high specific surface area of the Ca-MOF1 and the incomplete coordination of metal Ca, which can form coordination bonds with sulfur-containing functional groups in the fishy off-odor compounds.

### (2) Adsorption Energy

Based on the density functional theory (DFT) of first-principles, the adsorption energies of the Ca-MOF1 for different fishy off-odor compounds were calculated using the CASTEP module in Materials Studio software, with the formula as follows:

E=E_{adsorbent+adsorbate}−E_{adsorbent}−E_{adsorbate}

where E_{adsorbent} is the optimized energy of Ca-MOFs, E_{adsorbate} is the optimized energy of the fishy off-odor compounds, and E_{adsorbent+adsorbate} is the energy of the Ca-MOF after adsorbing the fishy off-odor compounds.

In addition, based on the molecular dynamics simulation theory, kinetic adsorption simulations were performed using a FORCITE module in the Materials Studio software to calculate the adsorption efficiencies of the Ca-MOF1 for the five types of fishy off-odor compounds.

TABLE 2

| | Adsorption energy E | Adsorption |
|---|---|---|
| | (kcal/mol) | efficiency (%) |
| Nonanal | −32.055 | 21.6 |
| 1-Octen-3-ol | −25.678 | 9.3 |
| Dimethyl trisulfide | −26.119 | 43.7 |
| Trimethylamine | −25.773 | 20 |
| 2-Methylisoborneol | −29.216 | 7.5 |

As shown in Table 2, according to DFT theoretical calculations, nonanal exhibits the highest adsorption energy (−32.055 kcal/mol), followed by 2-methylisoborneol (−29.216 kcal/mol), and then dimethyl trisulfide (−26.119 kcal/mol). Combining molecular dynamics simulations and experimental data analysis, dimethyl trisulfide shows the highest adsorption efficiency. Although nonanal and 2-methylisoborneol have relatively high adsorption energies, their large molecular sizes prevent them from being adsorbed within pores of the Ca-MOF, resulting in only surface adsorption. Adsorption sites inside the pores are occupied by small-molecule substances such as dimethyl trisulfide and trimethylamine. Dimethyl trisulfide can be adsorbed both on the surface and within the internal pores, thus exhibiting the highest adsorption efficiency. Therefore, the Ca-MOF1 is capable of selectively adsorbing dimethyl trisulfide.

To highlight the superior effects of the technical solution proposed in the present disclosure compared with the prior art, the following comparative experiments were conducted:

### Comparative Example 1

324 mg of γ-cyclodextrin and 112 mg of potassium hydroxide were accurately weighed, and dissolved in 10 mL of ultrapure water (a molar ratio of γ-cyclodextrin to potassium hydroxide was 1:8). The obtained solution was placed in a glass tube and ultrasonicated for 5 min to achieve uniform dispersion. After filtration through a 0.45 μm aqueous membrane, the solution was transferred to a small beaker containing 1 mL of methanol. The small beaker was then placed into a large beaker filled with methanol to allow the methanol to slowly diffuse into the solution in the small beaker and maintained at 50° C. for 6 h. The crystals were precipitated with methanol, and the MOF crystal precipitate was collected by centrifugation at 4000 rpm. The precipitate was washed twice with anhydrous methanol and dried overnight under vacuum at 45° C. to obtain a γ-cyclodextrin metal-organic framework (Y-CD-MOF1). (specificity in number)

### Comparative Example 2

260 mg of γ-cyclodextrin and 256 mg of potassium benzoate were accurately weighed, and dissolved in 10 mL of ultrapure water (a molar ratio of γ-cyclodextrin to potassium hydroxide was 1:8). The obtained solution was placed in a glass tube and ultrasonicated for 5 min to achieve uniform dispersion. After filtration through a 0.45 μm aqueous membrane, the solution was transferred to a small beaker containing 0.5 mL of methanol. The small beaker was then placed into a large beaker filled with methanol to allow the methanol to slowly diffuse into the solution in the small beaker, and was maintained at 50° C. for 6 h. The crystals were precipitated with methanol, and the MOF crystal precipitate was collected by centrifugation at 4000 rpm. The precipitate was washed twice with anhydrous methanol and dried overnight under vacuum at 45° C. A γ-cyclodextrin metal-organic framework (γ-CD-MOF2) was then obtained.

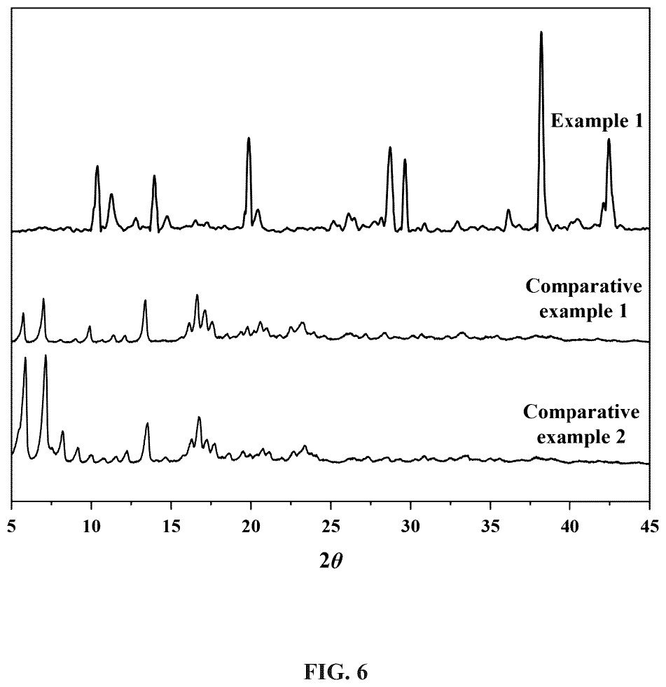

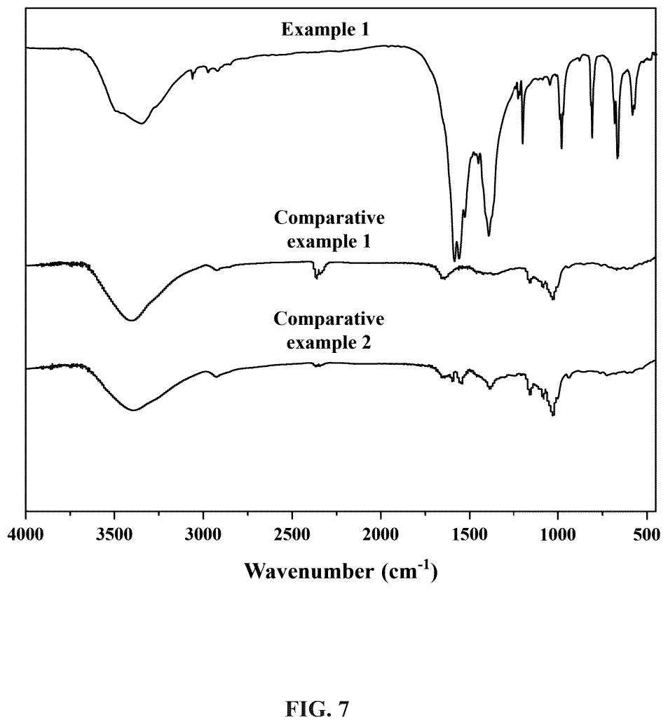

FIG. 6 shows the XRD results of the example and comparative examples, demonstrating that the Ca-MOF, the γ-CD-MOF1, and the γ-CD-MOF2 all exhibit good crystallinity; FIG. 7 shows the FTIR results of the example and comparative examples, with characteristic peaks indicating coordination between organic ligands and metal ions. The XRD and FTIR results indicate that all three crystalline materials were successfully synthesized.

Adsorption experiments were conducted, using the Ca-MOF1 from the present disclosure, the γ-cyclodextrin metal-organic framework (γ-CD-MOF1) from Comparative Example 1, and the γ-cyclodextrin metal-organic framework (γ-CD-MOF2) from Comparative Example 2, respectively, on the characteristic fishy off-odor compounds such as nonanal, 1-octen-3-ol, dimethyl trisulfide, trimethylamine, and 2-methylisoborneol. The adsorption efficiencies of the Ca-MOF, the γ-CD-MOF1, and the γ-CD-MOF2 for different types of fishy off-odor compounds are shown in Table 3.

TABLE 3

| | | Comparative | Comparative |
|---|---|---|---|
| | Example 1 | Example 1 | Example 2 |
| | (Ca-MOF1) | (γ-CD-MOF1) | (γ-CD-MOF2) |
| | Adsorption | Adsorption | Adsorption |
| | efficiency (%) | efficiency (%) | efficiency (%) |
| Nonanal | 85.7 | 58.2 | 55.4 |
| 1-Octen-3-ol | 47.1 | 46.9 | 39.1 |
| Dimethyl trisulfide | 97.3 | 80.3 | 78.7 |
| Trimethylamine | 79.2 | 60.1 | 53.2 |
| 2-Methylisoborneol | 42.5 | 46.7 | 57.3 |

As shown in Table 3, the Ca-MOF1 in the present disclosure exhibits a higher adsorption efficiency for dimethyl trisulfide than the γ-CD-MOF1 and the γ-CD-MOF2, and also demonstrates better adsorption effects for nonanal, 1-octen-3-ol, trimethylamine, and 2-methylisoborneol.

Based on Example 1, the following comparative experiments were conducted:

### Example 2

Accurately weighing 5 g of fumaric acid and 5 g of calcium carbonate, and then dissolving them in a mixed solution of methanol and ultrapure water (volume ratio 1:2), and fully dissolving under magnetic stirring; and transferring the solution into the polytetrafluoroethylene high-pressure reactor, reacting at 120° C. for 24 h, cooling to room temperature, collecting white crystals by centrifugation, washing 3 times with methanol, and drying under vacuum at 60° C. for 4 h to obtain a Ca-MOF2. A static adsorption experiment was conducted on the obtained Ca-MOF2. Table 4 shows the equilibrium adsorption capacities and adsorption efficiencies of the Ca-MOF2 for adsorbing five types of fishy off-odor compounds.

TABLE 4

| | Equilibrium adsorption | Adsorption |
|---|---|---|
| | capacity (μg/g) | efficiency (%) |
| Nonanal | 1397.31 | 73.1 |
| 1-Octen-3-ol | 1301.54 | 50.2 |
| Dimethyl trisulfide | 1852.29 | 78.5 |
| Trimethylamine | 8998.71 | 80.6 |
| 2-Methylisoborneol | 230.11 | 43.7 |

As shown in Table 4, after the calcium salt is replaced with calcium carbonate during the preparation of the Ca-MOF, the adsorption capacity for various types of fishy off-odor compounds decreases, and there is no selectivity in the adsorption of sulfur-containing substances.

Based on Example 1, different types of calcium salts were varied while keeping other conditions the same as in Example 1. The comparison results are shown in Table 5.

TABLE 5

| | Nonanal | | 1-Octen-3-ol | | Dimethyl trisulfide | | Trimethylamine | | 2-Methylisoborneol | |
|---|---|---|---|---|---|---|---|---|---|---|
| | | Adsorp- | | Adsorp- | | Adsorp- | | Adsorp- | | Adsorp- |
| | Equilibrium | tion | Equilibrium | tion | Equilibrium | tion | Equilibrium | tion | Equilibrium | tion |
| | adsorption | effi- | adsorption | effi- | adsorption | effi- | adsorption | effi- | adsorption | effi- |
| | capacity | ciency | capacity | ciency | capacity | ciency | capacity | ciency | capacity | ciency |
| | (μg/g) | (%) | (μg/g) | (%) | (μg/g) | (%) | (μg/g) | (%) | (μg/g) | (%) |
| Calcium | 1564.6 | 85.7 | 1209.7 | 47.1 | 2270.7 | 97.3 | 8936.3 | 79.2 | 220.1 | 42.5 |
| acetate | | | | | | | | | | |
| (Example 1) | | | | | | | | | | |
| Calcium | 1397.3 | 73.1 | 1301.5 | 50.2 | 1852.3 | 78.5 | 8998.7 | 80.6 | 230.1 | 43.7 |
| carbonate | | | | | | | | | | |
| (Example 2) | | | | | | | | | | |
| Calcium | 1189.7 | 62.5 | 1357.3 | 52.3 | 1698.5 | 72.4 | 8528.3 | 75.5 | 207.34 | 37.1 |
| chloride | | | | | | | | | | |
| Calcium | 1294.3 | 70.2 | 1108.3 | 42.7 | 1634.2 | 69.5 | 5835.7 | 51.7 | 184.2 | 33.2 |
| nitrate | | | | | | | | | | |

As shown in Table 5, different calcium salts have different effects on a pore structure of the Ca-MOF. When calcium acetate is used as the calcium salt, the prepared MOF material exhibits strong adsorption capacity for nonanal and dimethyl trisulfide. When calcium carbonate is used as the calcium salt, the prepared MOF material shows strong adsorption capacity for trimethylamine and 2-methylisoborneol but lacks selective adsorption ability. This may be due to the larger pores in the MOF material formed by calcium carbonate, which enhance the adsorption capacity for both small-molecule trimethylamine and larger-molecule 2-methylisoborneol, thereby reducing adsorption of other molecules. When calcium chloride is used as the calcium salt, the prepared MOF material exhibits strong adsorption capacity for 1-octen-3-ol. When calcium nitrate is used as the calcium salt, the prepared MOF material exhibits weaker adsorption capacity for all types of fishy off-odor compounds, possibly because of an incomplete MOF self-assembly structure and uneven pore diameter distribution.

### Example 3

Accurately weighing 5 g of fumaric acid and 5 g of calcium acetate, and then dissolving them in a mixed solution of methanol and ultrapure water (volume ratio 1:2), and fully dissolving under magnetic stirring; s and transferring the solution into the polytetrafluoroethylene high-pressure reactor, reacting at 65° C. for 24 h, cooling to room temperature, obtaining white crystals by centrifugation, washing 3 times with methanol, and drying under vacuum at 65° C. for 4 h to obtain a Ca-MOF3. A static adsorption experiment was conducted on the obtained Ca-MOF3. Table 6 shows the equilibrium adsorption capacities and adsorption efficiencies of the Ca-MOF3 for adsorbing five types of fishy off-odor compounds.

TABLE 6

| | Equilibrium adsorption | Adsorption |
|---|---|---|
| | capacity (μg/g) | efficiency (%) |
| Nonanal | 1409.32 | 76.2 |
| 1-Octen-3-ol | 1498.41 | 55.4 |
| Dimethyl trisulfide | 2024.13 | 86.7 |
| Trimethylamine | 9604.23 | 85.1 |
| 2-Methylisoborneol | 198.72 | 38.3 |

As shown in Table 6, when a reaction temperature in the MOF preparation process was changed from 120° C. to 65° C., the adsorption capacity for nonanal, dimethyl trisulfide, and 2-methylisoborneol decreases, while the adsorption capacity for 1-octen-3-ol and trimethylamine increases slightly. This indicates that the preparation temperature significantly affects the structure of the MOF material, and then affects the adsorption capacity.

Based on Example 1, different preparation reaction temperatures were varied, with all other conditions remaining the same as in Example 1. The comparison results are shown in Table 7.

TABLE 7

| | | | | | | | | | 2- | |
|---|---|---|---|---|---|---|---|---|---|---|
| | Nonanal | | 1-octen-3-ol | | Dimethyl trisulfide | | Trimethylamine | | methylisoborneol | |
| | | Adsorp- | | Adsorp- | | Adsorp- | | Adsorp- | | Adsorp- |
| | Equilibrium | tion | Equilibrium | tion | Equilibrium | tion | Equilibrium | tion | Equilibrium | tion |
| | adsorption | effi- | adsorption | effi- | adsorption | effi- | adsorption | effi- | adsorption | effi- |
| | capacity | ciency | capacity | ciency | capacity | ciency | capacity | ciency | capacity | ciency |
| | (μg/g) | (%) | (μg/g) | (%) | (μg/g) | (%) | (μg/g) | (%) | (μg/g) | (%) |
| 65° C. | 1409.3 | 76.2 | 1498.4 | 55.4 | 2024.1 | 86.7 | 9604.2 | 85.1 | 198.7 | 38.3 |
| (Example 3) | | | | | | | | | | |
| 95° C. | 1283.2 | 70.5 | 1284.4 | 49.8 | 1666.8 | 71.4 | 9291.3 | 82.2 | 217.3 | 40.1 |
| 120° C. | 1564.6 | 85.7 | 1209.7 | 47.1 | 2270.7 | 97.3 | 8936.3 | 79.2 | 220.1 | 42.5 |
| (Example 1) | | | | | | | | | | |
| 150° C. | 1159.6 | 63.2 | 1297.3 | 50.3 | 1653.2 | 70.2 | 8431.9 | 74.7 | 174.8 | 33.4 |

As shown in Table 7, different reaction temperatures have varying effects on the crystal structure of the Ca-MOF. The MOF material prepared at 65° C. exhibits strong adsorption capacity for dimethyl trisulfide and trimethylamine, although weaker than that of Example 1. This may be attributed to the formation of smaller pore diameters at this temperature compared to other temperatures; however, due to the pore structure and the flexibility of the framework, the adsorption capacity is inferior to that of Example 1. The MOF materials prepared at 95° C. and 150° C. show weak adsorption capacity for all types of fishy off-odor compounds, indicating that the MOF self-assembly structures formed at these two temperatures are incomplete with uneven pore diameter distribution.

### Example 4

Accurately weighing 5 g of fumaric acid and 5 g of calcium acetate, and then dissolving them in methanol (60 mL), and fully dissolving under magnetic stirring; and transferring the solution into the polytetrafluoroethylene high-pressure reactor, reacting at 120° C. for 24 h, cooling to room temperature, obtaining white crystals by centrifugation, washing 3 times with methanol, and drying under vacuum at 60° C. for 4 h to obtain a Ca-MOF4. A static adsorption experiment was conducted on the obtained Ca-MOF4. Table 8 shows the equilibrium adsorption capacities and adsorption efficiencies of the Ca-MOF4 for adsorbing five types of fishy off-odor compounds.

TABLE 8

| | Equilibrium adsorption | Adsorption |
|---|---|---|
| | capacity (μg/g) | efficiency (%) |
| Nonanal | 1197.63 | 65.7 |
| 1-Octen-3-ol | 1709.38 | 51.1 |
| Dimethyl trisulfide | 1532.90 | 65.2 |
| Trimethylamine | 7852.84 | 69.5 |
| 2-Methylisoborneol | 159.48 | 29.8 |

As shown in Table 8, variations in the ratio of the reaction solvent also affect the structure of the MOF. When methanol is used as the reaction solvent, the prepared MOF material exhibits higher adsorption capacity for 1-octen-3-ol. This indicates that different solvent ratios can influence the structure of the MOF material, which in turn affects the adsorption capacity.

Based on Example 1, the solvent-to-water ratio was varied while keeping all other conditions the same as in Example 1. The comparison results are shown in Table 9.

TABLE 9

| | | | | | | | | | 2- | |
|---|---|---|---|---|---|---|---|---|---|---|
| | Nonanal | | 1-Octen-3-ol | | Dimethyl trisulfide | | Trimethylamine | | Methylisoborneol | |
| | | Adsorp- | | Adsorp- | | Adsorp- | | Adsorp- | | Adsorp- |
| | Equilibrium | tion | Equilibrium | tion | Equilibrium | tion | Equilibrium | tion | Equilibrium | tion |
| | adsorption | effi- | adsorption | effi- | adsorption | effi- | adsorption | effi- | adsorption | effi- |
| | capacity | ciency | capacity | ciency | capacity | ciency | capacity | ciency | capacity | ciency |
| | (μg/g) | (%) | (μg/g) | (%) | (μg/g) | (%) | (μg/g) | (%) | (μg/g) | (%) |
| Methanol | 1197.6 | 65.7 | 1579.4 | 61.5 | 1532.9 | 65.2 | 7852.8 | 69.5 | 159.5 | 29.8 |
| (Example 4) | | | | | | | | | | |
| Ethanol | 1157.4 | 63.1 | 1551.3 | 60.4 | 1554.3 | 66.7 | 7939.4 | 70.6 | 122.7 | 23.7 |
| Water | 1487.5 | 80.4 | 1328.8 | 51.7 | 1998.4 | 85.5 | 5729.5 | 50.7 | 169.4 | 31.3 |
| Methanol:water = | 1564.6 | 85.7 | 1209.7 | 47.1 | 2270.7 | 97.3 | 8936.3 | 79.2 | 220.5 | 42.5 |
| 1:2 (Example 1) | | | | | | | | | | |
| Ethanol:water = | 1394.2 | 76.3 | 1059.3 | 41.3 | 1931.2 | 82.5 | 8111.7 | 71.2 | 201.1 | 38.6 |
| 1:2 | | | | | | | | | | |

As shown in Table 9, different solvent ratios also have varying effects on the structure of the Ca-MOF. When methanol or ethanol is used as the reaction solvent, the prepared MOF material exhibits strong adsorption capacity for 1-octen-3-ol in addition to adsorbing a large amount of trimethylamine. When pure water or ethanol-water (v: v=1:2) is used as the reaction solvent, the prepared MOF material shows weak adsorption capacity for the fishy off-odor compounds. This may be attributed to the low solubility of calcium acetate in the ethanol-water mixed solvent, which significantly affects the formation process of MOF self-assembly and thus influences the structure of the MOF.

Comparison Examples 2-4 and Tables 4-9 show that the type of calcium salt, reaction temperatures, and solvent ratios all affect the structure of the Ca-MOF, which in turn influences the adsorption capacity for the five different types of fishy off-odor compounds. The adsorption efficiency of the Ca-MOF1 prepared in Example 1 of the present disclosure for dimethyl trisulfide is much higher than that of Examples 2, 3, and 4, and the Ca-MOF1 has obvious selectivity, which indicates that the Ca-MOF1 has a selective adsorption effect on sulfur-containing fishy off-odor compounds.

### Example 5

Based on Example 1 and Comparative Example 1, a composite hybrid supramolecular MOF can be constructed using a nucleation kinetics-guided growth method. 3 g of γ-cyclodextrin and 1 g of potassium hydroxide were dissolved in 30 mL of ultrapure water and then placed in a glass tube and ultrasonicated for 5 min to achieve uniform dispersion. After filtration through a 0.45 μm aqueous membrane, the solution was transferred to a small beaker containing 10 mL of methanol. 1 g of Ca-MOF1 obtained in Example 1 was added. The small beaker was then placed into a large beaker filled with methanol to allow the methanol to slowly diffuse into the solution in the small beaker and maintained at 50° C. for 6 h. The crystals were precipitated with methanol, and the MOF crystal precipitate was collected by centrifugation at 4000 rpm. The precipitate was washed twice with anhydrous methanol and dried overnight under vacuum at 55° C. to obtain the composite hybrid supramolecular MOF. A static adsorption experiment was conducted on the obtained composite hybrid supramolecular MOF. Table 10 shows the equilibrium adsorption capacities and adsorption efficiencies for the five types of fishy off-odor compounds.

TABLE 10

| | Equilibrium adsorption | Adsorption |
|---|---|---|
| | capacity (μg/g) | efficiency (%) |
| Nonanal | 1581.52 | 86.2 |
| 1-Octen-3-ol | 2238.78 | 87.1 |
| Dimethyl trisulfide | 2261.81 | 97.0 |
| Trimethylamine | 9090.26 | 80.5 |
| 2-Methylisoborneol | 312.52 | 59.9 |

As shown in Table 10, the composite hybrid supramolecular MOF exhibits highly efficient adsorption capacity for the five types of fishy off-odor compounds. This may be attributed to the microporous, mesoporous, and macroporous structures, significantly expanding the application of the MOF material in the field of odor adsorption.

### Example 6

Adding 0.5 M of ammonium bicarbonate or ammonium carbonate to 50 mL of a saturated NaCl solution, stirring to dissolve, adding 1 M of fumaric acid at room temperature, and centrifuging and drying to form a fumaric acid monoamine salt; accurately weighing 5 g of fumaric acid monoamine salt, 5 g of fumaric acid and 10 g of calcium acetate, and then dissolving them in a mixed solution of methanol and ultrapure water (volume ratio 1:2), and fully dissolving under magnetic stirring; and transferring the solution into the polytetrafluoroethylene high-pressure reactor, reacting at 120° C. for 24 h, cooling to room temperature, obtaining white crystals by centrifugation, washing 3 times with methanol, and drying under vacuum at 60° C. for 4 h to obtain a Ca-MOF5. A static adsorption experiment was conducted on the obtained Ca-MOF5. Table 11 shows the equilibrium adsorption capacities and adsorption efficiencies of the Ca-MOF5 for adsorbing five types of fishy off-odor compounds.

TABLE 11

| | Equilibrium | | Equilibrium | | Equilibrium | |
|---|---|---|---|---|---|---|
| | adsorption | Adsorption | adsorption | Adsorption | adsorption | Adsorption |
| | capacity | efficiency | capacity | efficiency | capacity | efficiency |
| | (μg/g) | (%) | (μg/g) | (%) | (μg/g) | (%) |
| | 40° C. Example 1 | | 25° C. Example 1 | | 25° C. Example 6 | |
| Nonanal | 1564.61 | 85.7 | 1104.28 | 60.4 | 1136.25 | 62.2 |
| 1-Octen-3-ol | 1209.72 | 47.1 | 856.46 | 33.1 | 819.28 | 31.8 |
| Dimethyl | 2270.70 | 97.3 | 1741.83 | 74.6 | 2205.72 | 94.5 |
| trisulfide | | | | | | |
| Trimethylamine | 8936.26 | 79.2 | 9304.71 | 82.3 | 9647.82 | 85.7 |
| 2- | 220.01 | 42.5 | 171.57 | 33.2 | 158.94 | 30.6 |
| Methylisoborneol | | | | | | |

As shown in Table 11, the Ca-MOF5 has a significant impact on the adsorption capacity for sulfur-containing substances. Compared with 40° C., the Ca-MOF1 exhibits stronger adsorption capacity at 25° C. for trimethylamine with a low boiling point. However, after treatment in Example 6, the adsorption efficiency thereof was not significantly improved. On the contrary, the adsorption efficiency of dimethyl trisulfide was increased, possibly because the small molecular size of trimethylamine makes it difficult to be retained within the MOF pores. The enhanced polarity of the modified MOF material can provide more polar adsorption sites. Moreover, the flexibility and pore diameter of the MOF pores are more compatible with dimethyl trisulfide, allowing more dimethyl trisulfide molecules to be retained in the pores. This indicates that the treatment method in Example 6 can significantly enhance the adsorption capacity for sulfur-containing substances. Ammonium bicarbonate is not easily affected by the environment or reaction conditions, which helps maintain the stability of the reaction during synthesis. Moreover, the reaction conditions are mild. The decomposition of ammonium bicarbonate releases ammonia and carbon dioxide, which is beneficial for avoiding damage to the MOF structure.

### Example 7

Dissolving 0.5 M of ammonium bicarbonate or ammonium carbonate, 1.5 M of fumaric acid, and 2 M of Calcium acetate in 50 mL of saturated NaCl solution, then adding the solution to a mixed solution of methanol/ultrapure water (volume ratio 1:2), and fully dissolving under magnetic stirring; and transferring the solution into the polytetrafluoroethylene high-pressure reactor, reacting at 120° C. for 24 h, cooling to room temperature, obtaining white crystals by centrifugation, washing 3 times with methanol, and drying under vacuum at 60° C. for 4 h to obtain a Ca-MOF6. A static adsorption experiment was conducted on the obtained Ca-MOF6. Table 12 shows the equilibrium adsorption capacities and adsorption efficiencies of the Ca-MOF6 for adsorbing five types of fishy off-odor compounds.

TABLE 12

| | Equilibrium | | Equilibrium | | Equilibrium | |
|---|---|---|---|---|---|---|
| | adsorption | Adsorption | adsorption | Adsorption | adsorption | Adsorption |
| | capacity | efficiency | capacity | efficiency | capacity | efficiency |
| | (μg/g) | (%) | (μg/g) | (%) | (μg/g) | (%) |
| | 40° C. Example 1 | | 25° C. Example 6 | | 25° C. Example 7 | |
| Nonanal | 1564.61 | 85.7 | 1136.25 | 62.2 | 1120.87 | 61.4 |
| 1-Octen-3-ol | 1209.72 | 47.1 | 819.28 | 31.8 | 905.22 | 35.2 |
| Dimethyl | 2270.70 | 97.3 | 2205.72 | 94.5 | 2319.36 | 99.1 |
| trisulfide | | | | | | |
| Trimethylamine | 8936.26 | 79.2 | 9647.82 | 85.7 | 9422.27 | 83.5 |
| 2- | 220.01 | 42.5 | 158.94 | 30.6 | 168.08 | 32.7 |
| Methylisoborneol | | | | | | |

As shown in Table 12, the Ca-MOF directly modified with ammonium bicarbonate or ammonium carbonate exhibits strong adsorption capacity for sulfur-containing substances, but has little effect on nitrogen-containing substances, which further validates the conclusion of Example 6. Moreover, the treatment in Examples 6-7 can broaden the application range of the adsorbent.

The foregoing is merely intended to assist in understanding the method of the present disclosure and the essence thereof; however, the scope of protection of the present disclosure is not limited thereto. For those of ordinary skill in the art, within the technical scope disclosed by the present disclosure, any equivalent substitution or modification made according to the technical solution of the present disclosure and the inventive concept thereof shall be covered by the scope of protection of the present disclosure. In summary, the content of this specification should not be construed as a limitation to the present disclosure.

1-10. (canceled)

11. A preparation method of a Ca-MOF for selectively adsorbing fishy off-odor compounds, comprising the following steps:
S1. dissolving an organic acid and a calcium salt in an aqueous solution containing methanol, wherein the organic acid is fumaric acid, the calcium salt is calcium acetate, a volume ratio of the methanol to water is 1:2, and a molar ratio of the organic acid to the calcium salt is 1: (1˜5);
S2. transferring a fully dissolved solution into a high-pressure reactor for a high-temperature reaction at 120° C. for 6-72 h, and then cooling to room temperature; and
S3. performing centrifuging, alcohol washing, and drying on the cooled solution to obtain Ca-MOF crystals.

12. The preparation method of a Ca-MOF for selectively adsorbing fishy off-odor compounds according to claim 11, wherein the organic acid in step S1 is subjected to modification with an ammonium salt before use, and the ammonium salt is one or a combination of ammonium bicarbonate, ammonium carbonate, ammonium chloride, ammonium phosphate, and ammonium hydrogen phosphate, wherein a modification method comprises dissolving the ammonium salt and the organic acid in a saturated NaCl solution, with a molar ratio of the ammonium salt to the organic acid of 1: (1-5); and after forming a monoammonium salt of the organic acid, dissolving the monoammonium salt of the organic acid and the calcium salt together in the aqueous solution containing methanol.

13. The preparation method of a Ca-MOF for selectively adsorbing fishy off-odor compounds according to claim 11, wherein in step S1, an ammonium salt is further added to the organic acid and the calcium salt, wherein the ammonium salt is one or a combination of ammonium bicarbonate, ammonium carbonate, ammonium chloride, ammonium phosphate, and ammonium hydrogen phosphate, and a molar ratio of the ammonium salt to the organic acid to the calcium salt is 1: (1-5): (1-5).

14. The preparation method of a Ca-MOF for selectively adsorbing fishy off-odor compounds according to claim 11, wherein in step S3, alcohol washing is performed 1 to 5 times using ethanol or methanol, and drying conditions comprise vacuum drying at 20-100° C. for 1 to 24 h under a pressure of −0.01 to −0.1 MPa.

15. The preparation method of a Ca-MOF for selectively adsorbing fishy off-odor compounds according to claim 11, wherein the obtained Ca-MOF is involved in a preparation process of a γ-cyclodextrin metal-organic framework, and a composite supramolecular MOF is prepared by means of a nucleation kinetics-directed growth method, and then used for adsorption.

16. Uses of the Ca-MOF for selectively adsorbing fishy off-odor compounds obtained by the preparation method as per claim 11, wherein the Ca-MOF is used for selectively adsorbing the fishy off-odor compounds, and the fishy off-odor compounds comprise dimethyl trisulfide, nonanal, 1-octen-3-ol, trimethylamine, or 2-methylisoborneol.
